(19) United States
(12) Patent Application Publication
(10) Pub. No.: US 2015/0305779 A1
(43) Pub. Date: Oct. 29, 2015

(54) SPRING AND DEVICE FOR STABILIZING HUMAN OR ANIMAL BONE

(71) Applicant: Lorraine MONTAVON, Zürich (CH)

(72) Inventor: Lorraine MONTAVON, Zürich (CH)

(73) Assignee: Pierre M. Montavon, Zurich (CH)

(21) Appl. No.: 14/423,059

(22) PCT Filed: Aug. 21, 2012
(86) PCT No.: PCT/CH2012/000195

§ 371 Date: Jul. 15, 2015

(51) Int. Cl. A61B 17/70

(52) U.S. Cl. CPC A61B 17/7028; A61B 17/7001; A61B 17/7059

(57) ABSTRACT

Disclosed is a spring for a device for stabilizing bones. The spring is designed as a leg spring that includes a spring coil with at least one turn and, at the end, a first and a second leg, which project beyond the periphery of the turn. At its end, at least one leg includes a rod-shaped end piece that has a three-dimensional structure in the form of a turn or a peripheral leaf or lip. Also disclosed is a device for stabilizing bones, which includes a spring, a bone plate that includes means for securing the bone plate to the three-dimensional structure in the form of a turn or a peripheral leaf or lip, and a clamping element that has at least one hole for accepting a bone fixation element as well as a channel through which one of the two legs of the spring can be guided.

### BACKGROUND OF INVENTION

1. Field of Invention

The invention relates to a spring for stabilizing human or animal bones, to a device for stabilizing human or animal bones, and to a method for stabilizing long tubular bones or the vertebral column.

2. Brief Description of Related Art

A device for stabilizing the vertebral column is known from the document WO 02/102259 SENGUPTA. This device includes at least two pedicle screws, a spring element between the pedicle screws, and on each pedicle screw, a securing mechanism for securing the spring element to the pedicle screws. The spring element has a substantially straight end piece at each end and a central C-shaped or coil-shaped section arranged between the end pieces. On the straight end pieces, sleeves are attached, which increase the outer diameter of the end pieces, so that the end pieces can be secured in the pedicle screws. The securing mechanism consists of an open channel arranged at the end on the head of the pedicle screws for receiving an end piece of the spring element, a cap which is slid partially over the head and the end piece, and a nut by means of which the cap can be secured on the head of the pedicle screw and at the same time the end piece can be secured in the channel. Since, particularly in animals, in the lumbo-sacral portion of the vertebral column, only minimal space conditions for the implant exist, the voluminous form, in particular of the securing mechanism between the spring element and the pedicle screws of the aforementioned device, is disadvantageous.

An implant for the articulated connection of two vertebral bodies is known from US-A 2005/209694 LOEB. This known implant includes a spring element with a straight end piece at each end of the spring element and a central coil-shaped section. The end pieces each have a longitudinal hole for passing a bone securing element through it. However, the end pieces are connected rigidly to the central section, so that the bone fixation elements cannot be angled relative to a plane defined by the central axis of the coil-shaped section. The disadvantage of this embodiment therefore is that the bone securing elements as a result cannot be attached selectively in the pedicles and/or the laminas of the vertebral bodies, as a result of which the possibilities for the securing of the implant to the vertebral bodies are considerably reduced.

### BRIEF SUMMARY OF THE INVENTION

The purpose of the invention is to provide a remedy for this. The invention is based on the problem of producing a spring and a device for stabilizing bones, which allows a translational and rotational motion of at least one bone plate relative to the spring of the device, and thus allows the selective anchoring of the device by means of bone securing elements, for example, bone screws, in the laminas or in the pedicles of the vertebral bodies.

The invention solves the posed problem with a spring for stabilizing bones as disclosed and claimed herein, with a device for stabilizing bones as disclosed an claimed herein, and with a method for stabilizing long tubular bones or the vertebral column as disclosed and claimed herein.

The advantages achieved by the spring according to the invention are substantially that the spring can be made from a spring wire and a thread can be applied directly to the spring wire. As a result, an implant having a minimum volume is produced, so that the implant can be attached in the lumbo-sacral area of the vertebral column of dogs, for example, without limiting the movements of the animal. In addition, by means of a simple threaded connection, a bone plate can be attached to at least one leg, wherein, due to the translational and rotational mobility of the bone plate relative to the spring, the entire implant can be shortened, lengthened or twisted, so that the position of the bone securing element which can be introduced into the plate hole of the bone plate, for example, the position of a bone screw, can be selected after the introduction of the implant by the surgeon.

The advantages achieved by the device according to the invention are substantially that:
due to the translational and rotational movability of at least one bone plate relative to the spring, the device can be shortened, lengthened or twisted, so that the bone securing elements can be anchored in a desired position selectively in the pedicles and/or in the laminas of the vertebral bodies. As a result, the surgeon is given the possibility of setting the bone screws and the implant in such a manner that the bone screws are not pulled out even in the case of the great forces that occur in the lumbosacral area of the vertebral column, in particular in running or jumping dogs. Due to the threaded connection between at least one end piece of the spring and at least one bone plate, it becomes also possible to angle the bone plates and screws relative to the spring;
in the human and animal body, only a minimal space requirement (volume) for the device is needed;
relative to the spring of the device, axial slippage of the bone plates is not possible, even in the case of high stresses;
due to the threaded connection, a stable securing of the bone plate to the spring of the device is possible, even in the case of a small outer diameter of the end piece and a small bone plate;
the spring, in the case of a design as a leg spring, has a preferred swivel plane for the bone plates attached to the legs, so that the elastic resistance of the spring can be dimensioned for a defined movement, for example, extension and flexion of the vertebral bodies stabilized by the device, whereas considerably smaller or larger spring forces can act in the case of a torsion movement or a lateral bending of the vertebral bodies relative to one another; and
improvement, respectively promoting of callus formation in the case of long tubular bone fractures.

Additional embodiments of the invention can be commented on as follows:
The peripheral lamella or lip can have one or more threaded segments or, in an alternative embodiment, can be arranged in a plane perpendicular to the axis of the straight end piece. In further embodiments, several peripheral laminas or lips can be provided, which are arranged in several planes perpendicular to the axis of the straight end piece.
The first and second leg can comprise, at the end, a straight, preferably circular cylindrical end piece with a thread.
In a special embodiment, the spring coil has a variable pitch, which is implemented preferably in the form of only one turn of the spring coil. In an additional embodiment, the spring is produced from a spring wire which is wound around a central axis of the spring coil, and at least one leg in the direction of its free end is convergent relative to a plane that is perpendicular to the central axis, towards the other leg. In an additional embodiment, at least one leg is angled, relative to a plane perpendicular to the central axis, towards the other leg.

By means of these three embodiments, the variable pitch of one or both legs, in particular, can be arranged so that the straight end pieces come in contact substantially in a narrow axial region of the central axis of the spring coil. The bone securing means used for attaching the spring to bones can as a result be arranged in the immediate vicinity of a plane perpendicular to the central axis of the spring coil, so that the spring coil is stressed only in the turn direction upon bending and an unintentional deformation of the spring coil can be prevented.

Suitable materials for the spring are: stainless steel, nitinol, titanium, a titanium alloy, preferably TiAlNb, plastics, preferably fiber-reinforced plastics, or ceramic materials which allow a greater stiffness of the spring compared to plastics.

In an additional embodiment, the spring is produced from a hollow spring wire. The central fibers of the spring wire contribute nothing or only little to the bending stiffness, so that, due to the hollow formation, the bending stiffness is not decreased, but the dead weight is reduced.

In an additional embodiment, the spring is made from a spring wire having a preferably rectangular cross-sectional area that is flattened in the area of the spring coil. The advantage of this embodiment consists of the different bending stiffness of the spring wire in different directions.

In an additional embodiment, the spring coil is designed as a cone-shaped coiled spiral.

In an additional embodiment, the spring is made from a spring wire with a wire axis, and the sections of the wire axis, which are straight in the area of the first and second end piece of the first and second leg, enclose, viewed in a plane perpendicular to the central axis of the spring coil, an angle alpha which, in the unstressed state of the spring, is between 135° and 225°.

The advantage of this embodiment is that the end sections of the leg, which are provided for the attachment of the spring to the bone, can be aligned in a position that is suitable with regard to the anatomy.

In an additional embodiment, the leg spring can be prepared from a spring wire having a diameter d between 2.0 mm and 4.0 mm, preferably between 3.3 mm and 3.7 mm.

In an additional embodiment, the spring coil has several spring turns. Due to the larger number of spring turns, the leg spring can allow an increased relative movement of the vertebral body, under the same stress.

In an additional embodiment, the spring coil is made from a spring wire with a wire axis, wherein the wire axis of the spring wire coils in the area of the spring coil with a diameter D around the central axis, which is at least 12 mm, preferably at least 15 mm and typically 20 mm.

In a particular embodiment, the spring coil is made from a spring wire with a wire axis, wherein the wire axis of the spring coil coils in the area of the spring coil with a diameter D around the central axis, which is at most 25 mm, preferably at most 22 mm.

The invention moreover relates to a device for stabilizing bones in the human or animal body. In this device, the first and/or the second bone plate can have a cavity with an inner thread which can engage with the outer thread on the end piece of the first and/or the second leg. Each of the two bone plates can be moved towards the spring in translation or rotation relative to the longitudinal axis of the corresponding end piece. The bone plates can therefore be brought independently of one another each into an appropriate position on a vertebral body. The surgeon can therefore decide immediately before producing the bores for securing the bone securing elements in the bone, for example, in the vertebral bodies, whether the bone securing element should be anchored in the cortex, for example, in the lamina or in a pedicle of a vertebral body.

In an additional embodiment, the plate hole in the first and/or in the second bone plate has a hole axis, and the longitudinal axis of the cavity is arranged perpendicularly to the hole axis.

In an additional embodiment, the device includes a first and a second bone plate and, in addition, at least one clamping element with at least one hole for receiving a bone securing element and a channel for the passage of one of the two legs of the leg spring. The advantage of this embodiment is that the legs of the spring, in addition, can be attached to the bone surface, for example, to a vertebral body, in such a way that the leg spring can be secured against rotation. The device can be attached in such a manner to the bone, for example, to the vertebral bodies, that the bone screws are not pulled out, even in the case of great forces such as those that can occur in running or jumping dogs.

In an additional embodiment, at least one of the bone plates includes a first section having at least one plate hole, which is connected to an end piece of a leg, and a second section having at least one plate hole, wherein the second section is angled relative to the first section. The advantage of this embodiment is that the bone securing elements can be positioned optimally with regard to the anatomy.

In an additional embodiment, the plate hole, in the first section, has a hole axis, and the second section is angled relative to a plane orthogonal to the hole axis.

The first section and the second section can also be in a planar arrangement.

In an additional embodiment, the clamping element is designed as a clip, so that the end piece of a leg can be snapped into the channel in the clamping element. In this manner, the bone plates, at the end first, can be secured to the bone, and subsequently the clamping elements located at appropriate sites between the bone plates and the central section of the spring can be attached on or to the bone.

In an additional embodiment, the spring is made from a material that is different from the bone plates.

In addition, the device according to the invention can also comprise bone screws. The bone screws can each have a screw head which can be locked in a plate hole.

The device according to the invention is used for stabilizing vertebral bodies or for the temporary stabilization of a joint. For example, a joint on which a partial or complete abrasion of the collateral ligaments has taken place can be stabilized temporarily. Another use for the temporary stabilization of a joint exists in the case of bone defects resulting from the removal of a tumor.

The device according to the invention can moreover be used for treating a fractured bone and also for treating osteoarthritis or pseudarthrosis. The device according to the invention can be used preferably on the knee joint or on finger joints.

The spring according to the invention can also be used for treating a fractured bone, or osteoarthritis or pseudarthrosis, wherein the spring is used for the external securing.

The spring can be attached, for example, to a plaster cast or it can be attached in the form of an external fixation by means of bone securing elements to the bone.

The invention also relates to a method for stabilizing a long tubular bone or the vertebral column, having the steps of:
i) producing an incision in the area of the bone section to be treated, more specifically a dorsal or posterior incision in the area of the vertebral bone section;
ii) preparing the epaxial muscles and the osseous attachment sites for securing the implant;
iii) performing a lateral retraction of the muscle masses;
iv) introducing an implant through the incision;
v) securing the implant to the bone section, or to the vertebral bodies, and
vi) closing the incision.

In a particular embodiment of the method according to the invention, step i) is preceded by the following additional steps:
diagnosing the degree and type of compression by means of an imaging investigation and/or
determining the maximum stretching and bending of the vertebral column segment to be treated.

In an additional embodiment of the method according to the invention, after step v), the additional step of:
performing a surgical decompression within the vertebral column channel can be carried out.

In the method according to the invention, a device according to the invention can be implanted.

### BRIEF DESCRIPTION OF THE DRAWINGS

The invention and variants of the invention are explained in further detail below in reference to partially diagrammatic representations of several embodiment examples.

FIG. 1 shows a side view of an embodiment of the device according to the invention implanted on a lumbosacral vertebral column section of a dog in a side view;

FIG. 2 shows a top view of an additional embodiment of the devices according to the invention implanted on a lumbosacral vertebral column section of a dog in a dorsal view;

FIG. 3 shows a side view of an embodiment of the spring according to the invention;

FIG. 4 shows a top view of the embodiment of the spring according to the invention according to FIG. 3;

FIG. 5 shows a side view of a bone plate according to the embodiment of the device according to the invention according to FIG. 1;

FIG. 6 shows a top view of the bone plate according to the embodiment of the device according to the invention according to FIG. 1;

FIG. 7 shows a section through a clamping element according to the embodiment of the device according to the invention according to FIG. 2;

FIG. 8 shows a top view of a clamping element according to the embodiment of the device according to the invention according to FIG. 2;

FIG. 9 shows a top view of a bone plate according to another embodiment of the device according to the invention;

FIG. 10 shows a side view of a bone plate according to yet another embodiment of the device according to the invention;

FIG. 11 shows a side view of a bone plate according to an additional embodiment of the device according to the invention;

FIG. 12 shows a view of another embodiment of the device according to the invention implanted on a tubular bone; and

FIG. 13 shows a top view of a spring according to another embodiment of the device according to the invention.

### DETAILED DESCRIPTION OF THE INVENTION

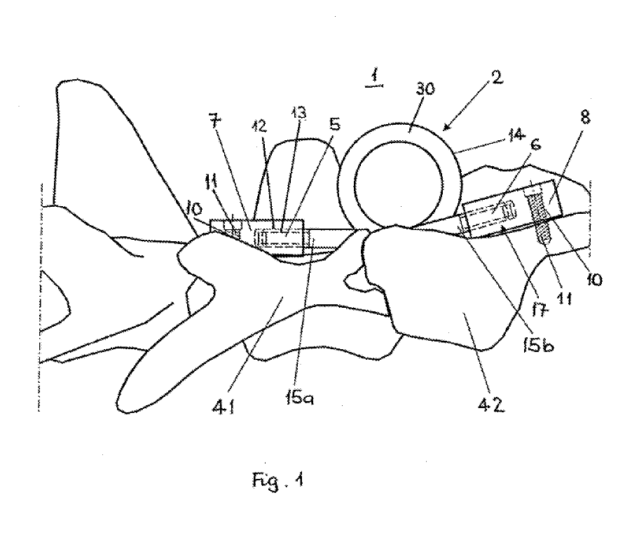

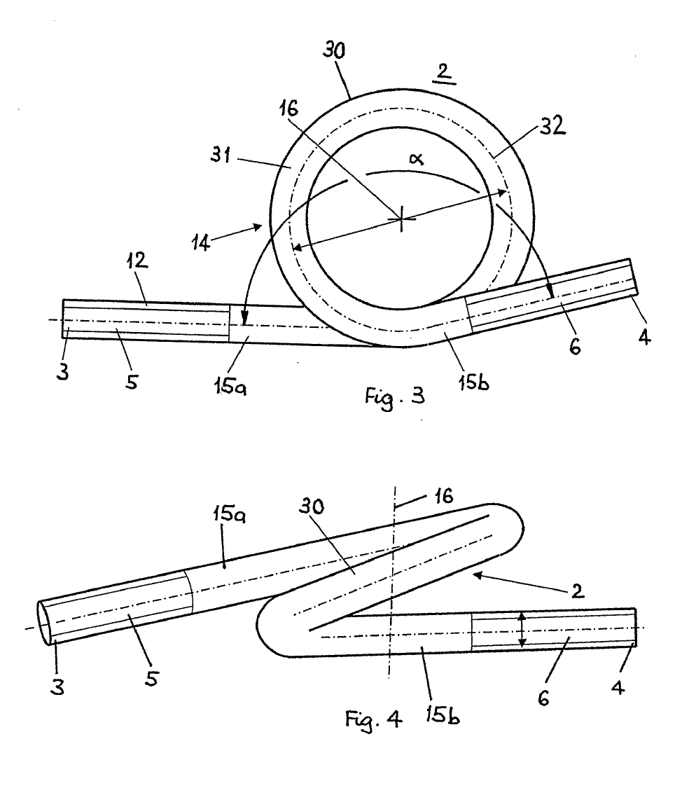

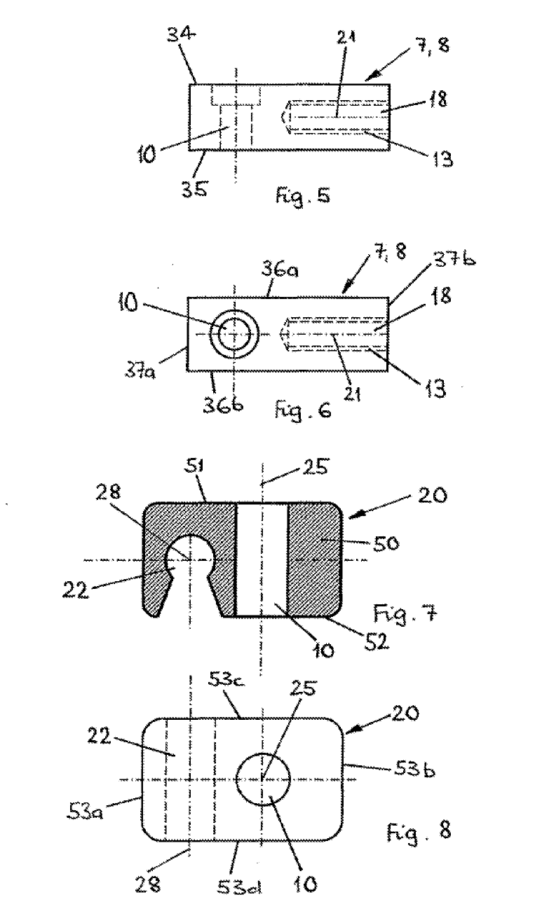

FIG. 1 is a representation of an embodiment of the device 1 according to the invention for stabilizing the vertebral column, for example, the last lumbar vertebral body 41 relative to the sacrum 42, for example, of a dog, wherein each device 1 is arranged on one side of the spinous processes of the vertebral body. Each of the two devices 1 includes a spring 2 (FIGS. 3 and 4) which is designed as leg spring 14 and which, at the end, includes a first leg and a second leg 15a, 15b, a first bone plate 7 arranged on the first leg 15a (FIGS. 5 and 6), and a second bone plate 8 arranged on the second leg 15b (FIGS. 5 and 6). The first bone plate and the second bone plate 7, 8 each have a plate hole 10 for receiving a bone securing element 11, so that the spring 2 can be attached by means of the bone fixation element 11 inserted in the plate holes 10 of the first and second bone plates 7, 8, for example, on the last lumbar vertebral body 41 and on the sacrum 42. The first leg and the second leg 15a, 15b each have an end piece 5, 6, each of which is connected by means of a threaded connection 17 to one of the bone plates 7, 8.

By securing the spring 2, for example, on the last lumbar vertebral body 41 and on the sacrum 42, a resistance is opposed by the spring 2 against the great forces occurring in the lumbosacral area of the vertebral column, particularly in running and jumping dogs, so that painful impacts or contacts between the last lumbar vertebral body 41 and the sacrum 42 can be prevented.

Brief description of the implantation of the device according to the invention:

First, the degree and the type of the compression of the spinal cord are diagnosed by an imaging investigation. In addition, the maximum stretching and bending of the vertebral column segment to be treated can subsequently be determined. For the introduction, positioning and securing of the device, the dorsal or posterior access is used. Before that, a bilateral preparation of the epaxial muscles and a surgical preparation of the osseous attachment sites are carried out. The lateral retraction of the muscle masses is established by the self-holding retractors. The pedicle, the transverse processes and the spinous processes are now exposed and they allow the securing of the implant with bone securing elements, preferably with bone screws having the necessary different angles relative to the device. A surgical decompression within the vertebral column channel is then also possible. After securing the implant, the incision is closed again.

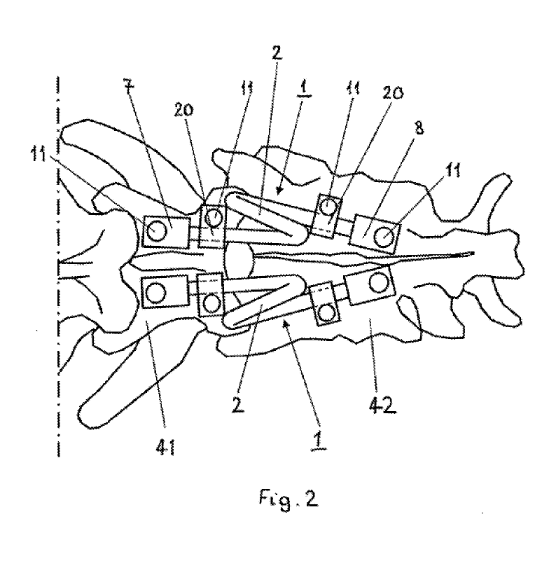

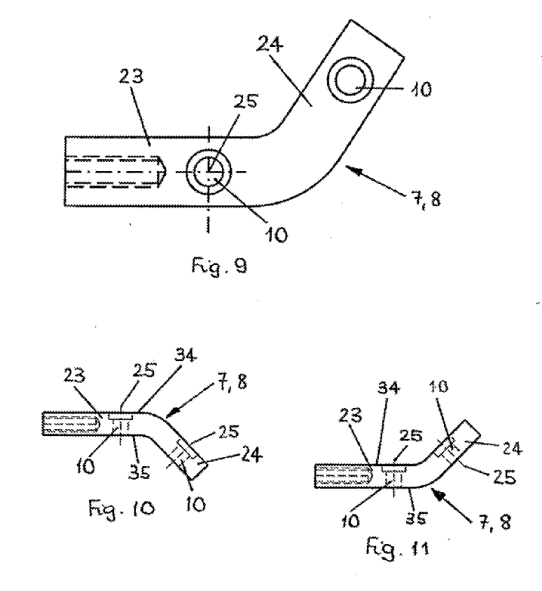

FIG. 2 shows a use of another embodiment of the device 1 according to the invention, also in the lumbosacral area of the vertebral column, which differs from the embodiment according to FIG. 1 only in that each of the two devices 1 includes, in addition, a clamping element 20 (FIGS. 7 and 8), each being attached to one of the legs 15a, 15b and secured by means of an additional bone fixation element 11 to the last lumbar vertebral body 41 and on the sacrum.

In FIGS. 3 and 4, an embodiment of the spring 2 is represented. The spring 2 is designed as a leg spring 14, and it includes a spring coil 30 coiled around a central axis 16, a first end 3 and a second end 4. The spring coil 14 is made from a spring wire 31 and includes in each case a preferably circular cylindrical leg 15a, 15b arranged at the end. The spring coil 30 has, for example, one spring turn. The spring wire 31 can have a circular cylindrical design with a diameter d. The wire axis 32 of the spring wire 31 is coiled in the area of the spring coil 30 with a diameter D in a helical pattern around the central axis 16 of the spring coil 30, and at the transitions, it transitions tangentially towards the legs 15a, 15b into a respective straight section in the area of the legs 15a, 15b. The leg spring 14 is also referred to as screw-like coiled bending springs, wherein a torque exerted on the legs 15a, 15b acts around the central axis 16 of the spring coil 30 as a substantially constant torque on the spring wire 31 in the entire area of the spring coil 30. The projections of the sections of the wire axis 32 of the spring wire 31, which are straight in the area of the legs 15a, 15b, into a plane perpendicular to the central axis 16 of the spring coil 30, enclose an angle alpha which, in the unstressed state of the spring 2, can be between approximately 135° and approximately 225°, and which in the present embodiment is approximately 165°, for example. The legs 15a, 15b thus form levers, which can be turned relative to one another against the elastic resistance of the spring 2, so that the angle alpha is increased or decreased. When the legs 15a, 15b are rotated under the action of a force relative to one another, the spring wire 31 is bent in the area of the spring coil 30, i.e. inward or outward. The spring 2 is made from a material that is different from the first and the second bone plate 7, 8 and can be made from a titanium alloy, preferably from TiAlNb, a plastic, a ceramic material or from nitinol. Furthermore, the legs 15a, 15b, on their end pieces 5, 6, each have a thread 12, so that the legs 15a; 15b of the spring 2 can be secured in a rigid manner on the first and second bone plates 7, 8 (FIGS. 5 and 6). The diameter d of the spring wire 31 can be between 2.0 mm and 4.0 mm. A typical value for the diameter d is 3.5 mm. In the area of the spring coil 30, the diameter D enclosed by the wire axis 32 of the spring wire 31 is typically 20 mm.

The spring coil 30 has a variable pitch, so that on the first end and the second end 3, 4 of the spring 2, the wire axis 32 of the spring wire 31 is located substantially in a plane perpendicular to the central axis 16 of the spring coil 30. In this way, it is possible to achieve that the straight end pieces 5, 6 of the first and of the second leg 15a, 15b come to be located substantially in a narrow axial area of the central axis 16 of the spring coil 30.

As shown in FIGS. 5 and 6, the first and the second bone plates 7, 8 have a cuboid design and they each comprise a top side 34, a bottom side 35, two long side surfaces 36a, 36b and two short side surfaces 37a, 37b. Each of the first and second bone plates 7, 8 includes a plate hole 10 which passes through the first bone plate and the second bone plate 7, 8 from the top side 34 to the bottom side 35. Moreover, each one of the first and second bone plates 7, 8 comprises a cavity 18 penetrating from a short side surface 37b into the bone plate 7, 8. The cavity 18 is designed as a blind hole with a longitudinal axis 21 which is perpendicular to the hole axis 25 of the plate bore 10, and it has an inner thread 13 which matches the outer threads 12 on the end pieces 5, 6 of the legs 15a, 15b, so that in each case one of the first and second bone plates 7, 8 can be secured by means of a threaded connection 17 to an end piece 5, 6 of a leg 15a, 15b. The first and second bone plates 7, 8 can be produced from a biocompatible stainless steel, for example, an austenitic stainless chromium-nickel-molybdenum steel, titanium or a titanium alloy, for example, Ti-6Al-7Nb.

FIGS. 7 and 8 show an embodiment of the clamping element 20 (FIG. 2). The clamping element 20 includes a three-dimensional body 50, which is produced, for example, as a cuboid body having a top side 51, a bottom side 52 and four side walls 53a, 53b, 53c, 53d. The clamping element 20 is designed as an elastic clip and includes a plate hole 10 having a hole axis 25, hole which passes through the three-dimensional body 50, and a channel 22 having a hole axis 25 skewed relative to the channel axis 28, hole which also passes through the three-dimensional body 50. The channel 22 is suitable for receiving a leg 15a, 15b of the leg spring 14 and includes a channel wall that is open at the periphery towards the bottom side 52 of the clamping element 20. The hole axis 25 and the channel axis 28 are a distance apart which is measured so that plate hole 10 and the channel 22 do not interpenetrate. The plate hole 10 passes through the three-dimensional body 50 from the top side 51 thereof to the bottom side 52, while the channel 22 passes through the three-dimensional body 50 from a first side wall 53c to a facing second side wall 53d. The channel 22 narrows towards the bottom side 52 of the clamping element 20 in such a manner that a leg 15a, 15b of a spring 2 can be snapped into the channel 22 from the bottom side 52 of the clamping element 20. If the spring 2 has to be secured by means of an additional clamping element 20 to the vertebral column, then an additional clamping element 20 can be simply placed on said vertebral column, without having to be slid painstakingly over the length of the legs 15a, 15b of the spring 2. Furthermore, the spring 2 can be introduced into the human or animal body before the setting of the bone securing elements 11, so that the positions of the bone fixation elements to be secured subsequently on the vertebral bodies can be determined easily by the surgeon.

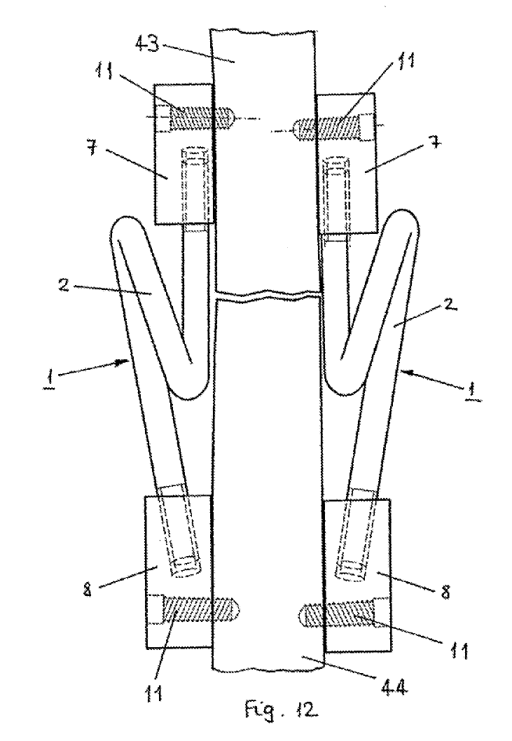

In FIGS. 9 to 11, additional embodiments of the bone plates 7, 8 are represented, which differ from the embodiment represented in FIGS. 5 and 6 only in that the first and/or the second bone plate 7, 8 comprise(s), in addition to a first section 23 having a plate hole 10, which can be connected to an end piece 5, 6 of a leg 15a, 15b, a second section 24 having a plate hole 10, wherein the second section 24 is angled relative to the first section 23. FIG. 9 shows an embodiment of the first and/or the second bone plate 7, 8, wherein the first section and the second section 23, 24 are in a planar arrangement. In the embodiment of the first and/or the second bone plate 7, 8 shown in FIG. 10, the first section and the second section 23, 24 are angled relative to one another so that the hole axis 25 of the plate hole 10 in the first section 23 and the hole axis 25 of the plate hole 10 in the second section 24 converge towards the bottom side 35 of the first and/or the second bone plate 7, 8, while in the embodiment of the first and/or the second bone plate 7, 8 represented in FIG. 11, the first section and the second section 23, 24 are angled with respect to one another in such a manner that the hole axis 25 of the plate hole 10 in the first section 23 and the hole axis 25 of the plate hole 10 in the second section 24 diverge towards the bottom side 35 of the first and/or the second bone plate 7, 8.

The embodiments of the bone plates 7, 8 represented in FIGS. 9 to 11 are suitable as well for the stabilization of vertebral column segments or of joints.

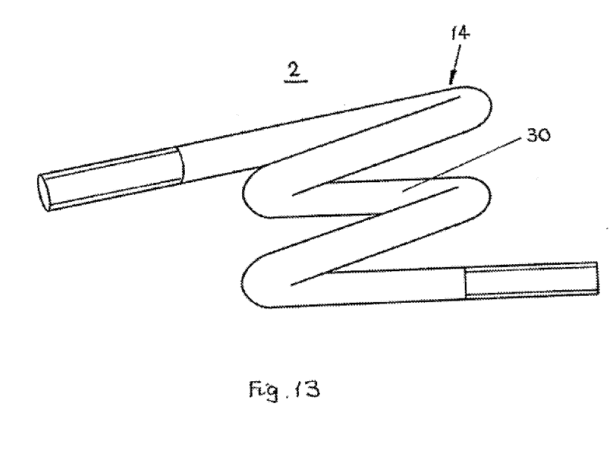

In FIG. 12, a use of the device 1 according to the invention for treating a fractured bone is represented. Here, a respective device 1 is arranged on the medial and on the lateral side of the fractured bone. Each one of the two devices 1 includes a spring 2 whose first leg 15a is connected by means of a threaded connection 17 to a first bone plate 7, wherein the two first bone plates 7 are each secured with a bone securing element 11 to the proximal bone fragment 43. Similarly, the second legs 15b of the spring 2 are each connected by means of a threaded connection 17 to a second bone plate 8, wherein the second bone plates 8 are each attached to a bone securing element 11 on the distal bone fragment 44. If the surgeon so desires, the springs 2 arranged on the medial side and the lateral side of the bone fragments 43, 44 can have a different spring constant and, in addition, one or more clamping elements 20 (FIGS. 7 and 8) can be mounted on the springs 2.

The embodiment of the spring 2 represented in FIG. 12 differs from the embodiment represented in FIGS. 3 and 4 only in that the spring 2 designed as a leg spring 14 has several spring turns in its helical section 30.

Although, as described above, different embodiments of the present invention are present, they should be understood in such a way that the different features can be used both individually and also in any desired combination.

The invention is therefore not limited to the above-mentioned particularly preferable embodiments.

1. A spring for stabilizing bones, wherein the spring is designed as leg spring, which includes a spring coil with at least one turn and, a first leg having an end and a second leg having an end, wherein each of said first leg and said second leg projects beyond a periphery of the turn,
wherein
the end of at least one of said first leg and second leg, includes a rod-shaped end piece, which has a three-dimensional structure in the form of a thread or a peripheral lamella or lip.

2-9. (canceled)

10. The spring according to claim 1, wherein the spring coil includes several spring turns.

11. A device for stabilizing bones in human or animal body, comprising:
a spring according to claims 1; and
a bone plate having at least one plate hole for receiving a bone securing element and means for securing the bone plate to one of the first leg or the second leg, which means can engage with the three-dimensional structure; and wherein the device additionally comprises:
clamping element having at least one hole for receiving a bone securing element and a channel for the passage of the other one of the first leg or second leg of the spring,
or
a second bone plate having at least one plate hole for receiving a bone securing element and means for securing the bone plate to the other one of the first leg and the second leg, which can engage with the three-dimensional structure.

12. The device according to claim 11, wherein the first and/or the second bone plate include(s) a cavity with an inner thread, which can engage with the outer thread on the end piece of the first and/or second leg.

13. The device according to claim 12, wherein the plate hole in the first and/or second bone plate has a hole axis, and a longitudinal axis of the cavity is arranged perpendicular to the hole axis.

14. The according to claims 11, wherein the device has a first and a second bone plate and, in addition, at least one clamping element having at least one hole for receiving a bone securing element and a channel for the passage of one of the first leg or the second leg of the leg spring.

15. The device according to claim 11, wherein at least one of the bone plates includes a first section having at least one plate hole, which is connected to the end piece of the first leg or second leg, and a second section having at least one plate hole, wherein the second section is angled relative to the first section.

16. The device according to claim 15, wherein the plate hole in the first section has a hole axis, and the second section is angled relative to a plane orthogonal to the hole axis.

17. (canceled)

18. The device according to claim 11, wherein the clamping element is designed as a clip, so that the end piece of one of the first leg or the second leg can be snapped into the channel in the clamping element.

19. The device according to claim 11, wherein the spring is made from a material that is different from the bone plates.

20. The device according to claim 11, wherein the device additionally includes bone screws.

21-25. (canceled)

26. A method for stabilizing a long tubular bone or a vertebral column, comprising:
producing an incision in an area of a bone section to be treated;
preparing epaxial muscles and osseous attachment sites for securing an implant;
performing a lateral retraction of muscle masses;
introducing the implant through the incision;
securing the implant on the bone section, and
closing the incision.

27. The method according to claim 26, further comprising, prior to producing the incision, diagnosing the degree and the type of compression by means of an imaging investigation.

28. The method according to claim 26, further comprising, prior to producing the incision, determining the maximum stretching and bending of the vertebral column segment to be treated.

29. The method according to one of claim 26, further comprising, after securing the implant on the bone section, performing a surgical decompression within the vertebral column channel.

30. The method according to claim 26, wherein the implant is a device according to claim 11.
